(19) United States
(12) Patent Application Publication
(10) Pub. No.: US 2026/0216525 A1
(43) Pub. Date: Jul. 30, 2026

(54) CUSTOMIZING CARDIAC ARREST TREATMENT DURING CPR

(71) Applicant: KONINKLIJKE PHILIPS N.V., EINDHOVEN (NL)

(72) Inventors: DAWN BLILIE JORGENSON, Bothell, WA (US); STACY EARL GEHMAN, Bothell, WA (US); CHENGUANG LIU, Bothell, WA (US)

(21) Appl. No.: 19/144,666

(22) PCT Filed: Dec. 21, 2023
(86) PCT No.: PCT/EP2023/087164

§ 371 Date: Jun. 30, 2025

Related U.S. Application Data
(60) Provisional application No. 63/436,517, filed on Dec. 31, 2022.

(51) Int. Cl. A61N 1/39; A61N 1/372

(52) U.S. Cl. CPC A61N 1/3925; A61N 1/37247; A61N 1/39044

(57) ABSTRACT

A defibrillator for customizing a cardiac therapy treatment of a patient during a cardiopulmonary resuscitation (CPR) being administered by a responder to a heart of the patient. In operation, defibrillator monitors an ECG waveform of the heart of the patient during the cardiopulmonary resuscitation being administered by a responder to the heart of the patient. When the ECG waveform as monitored by the defibrillator indicates a shockable rhythm of the heart of the patient during the CPR being administered by a responder to the heart of the patient, the defibrillator derives a treatment advisory from a quality of the CPR being administered by the responder to the heart of the patient and a vitality of the shockable rhythm of the heart of the patient, and optionally from other factors.

### FIELD OF THE INVENTION

The present disclosure generally relates to a cardiac arrest treatment involving cardiopulmonary resuscitation (“CPR”) being administered by a responder to a heart of a patient, and more particularly to customizing the cardiac arrest treatment to the patient during the CPR.

### BACKGROUND OF THE INVENTION

FIG. 1 illustrates a CPR monitor 30 positioned on the sternum of a patient 10 as a responder 20 applies chest compressions in a conventional manner using two hands with one placed over the other. Instead of placing the hands directly on the patient 10, however, the hands of responder 20 are placed on the CPR monitor 30 and chest compressions are applied to the patient 10 via the CPR monitor 30. Chest compressions are administered by the responder 20 to a heart of patient 10 as prescribed by conventional CPR protocols. As known in the art of the present disclosure, the CPR monitor 30 monitors a quality of the CPR being administered by a responder 20 to a heart of patient 10, such as, for example, whether the CPR is effective or ineffective in terms of a depth and a rate of compression, chest release and recoil, and placement of the responder's hands on the chest of patient 10. A cable 31 is attached to a defibrillator 40 to couple the monitoring of the CPR quality to defibrillator 40 and to issue audible CPR instructions through a loudspeaker of defibrillator 40.

FIG. 1 further illustrates defibrillator 40 attached to patient 10 by electrodes 41a and 41b. Defibrillator 40, as known in the art of the present disclosure, is used to deliver defibrillating shocks to the patient 10 during the CPR as needed. More specifically, defibrillator 40 is operable to deliver a high-voltage impulse to a heart of patient 10 in order to restore normal rhythm and contractile function in patients who are experiencing an arrhythmia (e.g., ventricular fibrillation (VF) or ventricular tachycardia (VT)) that is not accompanied by spontaneous circulation. In operation, defibrillator 20 automatically analyzes an electrocardiogram (ECG) rhythm of the heart of patient 10 to determine if defibrillation is necessary. If so, defibrillator 40 prompts responder 20 to terminate the CPR and to press a shock button to deliver the defibrillation shock to the patient when a shock is advised by defibrillator 40.

The field of resuscitation, as exemplarily shown in FIG. 1, is heavily focused on increasing a quality of care by identifying and providing optimal CPR/shock treatment for a patient experiencing cardiac arrest.

### SUMMARY OF THE INVENTION

The present disclosure is directed to customizing a cardiac therapy treatment of a patient during a cardiopulmonary resuscitation (CPR) being administered by a responder to a heart of the patient by deriving and communicating a treatment advisory from an evaluation of a quality of the CPR by the responder (e.g., in terms of depth and rate of compressions, chest release and recoil, and placement of a responder's hands on a patient's chest) in combination with a determination of a vitality of a detected shockable rhythm of the heart of the patient (e.g., ventricular fibrillation (VF) or ventricular tachycardia (VT)).

The treatment advisory may audibly, visually, textually or graphically instruct the responder to a continue, modify or interrupt the CPR being administered by the responder to the heart of the patient.

The treatment advisory may audibly, visually, textually or graphically instruct the responder to deliver a shock to the heart of the patient.

The present disclosure may be embodied as (1) a defibrillator, (2) a defibrillation controller and (3) a defibrillation method.

Various exemplary embodiments of a defibrillator of the present disclosure encompass an ECG monitor and a defibrillation controller for customizing a cardiac therapy treatment of a patient during CPR being administered by a responder to a heart of the patient. The ECG monitor is configured to monitor an ECG waveform of the heart of the patient during the CPR being administered by the responder to the heart of the patient. The defibrillation controller is configured to, when the ECG waveform as monitored by the ECG monitor indicates a shockable rhythm of the heart of the patient during the CPR being administered by the responder to the heart of the patient, derive a treatment advisory from at least a quality of the CPR being administered by the responder to the heart of the patient and a vitality of the shockable rhythm of the heart of the patient. The defibrillation controller is further configured to customize the cardiac therapy treatment to the patient by communicating the treatment advisory to the responder.

Various exemplary embodiments of a defibrillation controller of the present disclosure encompass a non-transitory machine-readable storage medium encoded with instructions for execution by one or more processors to adapting a cardiac therapy treatment of a patient during CPR being administered to a heart of the patient. The exemplary non-transitory machine-readable storage medium includes instructions to (1) when an ECG waveform indicates a shockable rhythm of the heart of the patient during the CPR being administered by a responder to the heart of the patient, derive a treatment advisory from at least a quality of the CPR being administered to the patient and a vitality of the shockable rhythm of the heart of the patient and (2) customize the cardiac therapy treatment to the patient by communicating the treatment advisory to the responder.

Various exemplary embodiments of a defibrillation methods in accordance with the present disclosure encompass adapting a cardiac therapy treatment of a patient during CPR being administered to a heart of the patient by (1) monitoring, by a defibrillator, a ECG waveform of the heart of the patient during the cardiopulmonary resuscitation being administered to a heart of the patient, (2) deriving, by the defibrillator when the ECG waveform as monitored by the defibrillator indicates a shockable rhythm of the heart of the patient during the CPR being administered by a responder to the heart of the patient, a treatment advisory from at least a quality of the CPR being administered to the patient and a vitality of the shockable rhythm of the heart of the patient, and (3) customizing the cardiac therapy treatment to the patient by the defibrillator communicating the treatment advisory to the responder.

The foregoing exemplary embodiments and other embodiments of the present disclosure as well as various structures and advantages of the present disclosure will become further apparent to those having ordinary skill in the art from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

The present disclosure will present in detail the following description of exemplary embodiments with reference to the following figures wherein:

FIG. 1 illustrates a cardiopulmonary resuscitation being administered by a responder to a heart of the patient as known in the art of the present disclosure;

FIG. 2 illustrates an exemplary embodiment of a cardiac arrest treatment system in accordance with the present disclosure;

FIG. 3 illustrates an exemplary embodiment of a defibrillator in accordance with the present disclosure;

FIG. 4 illustrates a flowchart representative of an exemplary embodiment of a pre-shock defibrillation method in accordance with the present disclosure;

FIGS. 5A-5D illustrate exemplary treatment advisory communications in accordance with FIG. 4 of the present disclosure;

FIG. 6 illustrates a flowchart representative of an exemplary embodiment of a post-shock defibrillation method in accordance with the present disclosure;

FIG. 7A illustrates an exemplary ECG waveform of a refractory ventricular fibrillation as known in the art of the present disclosure.

FIG. 7B illustrates an exemplary ECG waveform of a recurrent ventricular fibrillation as known in the art of the present disclosure.

FIGS. 8A and 8B illustrate exemplary treatment advisory communications in accordance with FIG. 6 of the present disclosure; and

FIG. 9 illustrates an exemplary embodiment of a defibrillation controller in accordance with the present disclosure.

### DETAILED DESCRIPTION OF THE PREFERRED EMBODIMENTS

The present disclosure is directed to customizing a cardiac therapy treatment of a patient during a cardiopulmonary resuscitation (CPR) being administered by a responder to a heart of the patient by deriving and communicating a treatment advisory from an evaluation of a quality of the CPR by the responder (e.g., in terms of depth and rate of compressions, chest release and recoil, and placement of a responder's hands on a patient's chest) in combination with a determination of a vitality of a detected shockable rhythm of the heart of the patient (e.g., ventricular fibrillation (VF) or ventricular tachycardia (VT)).

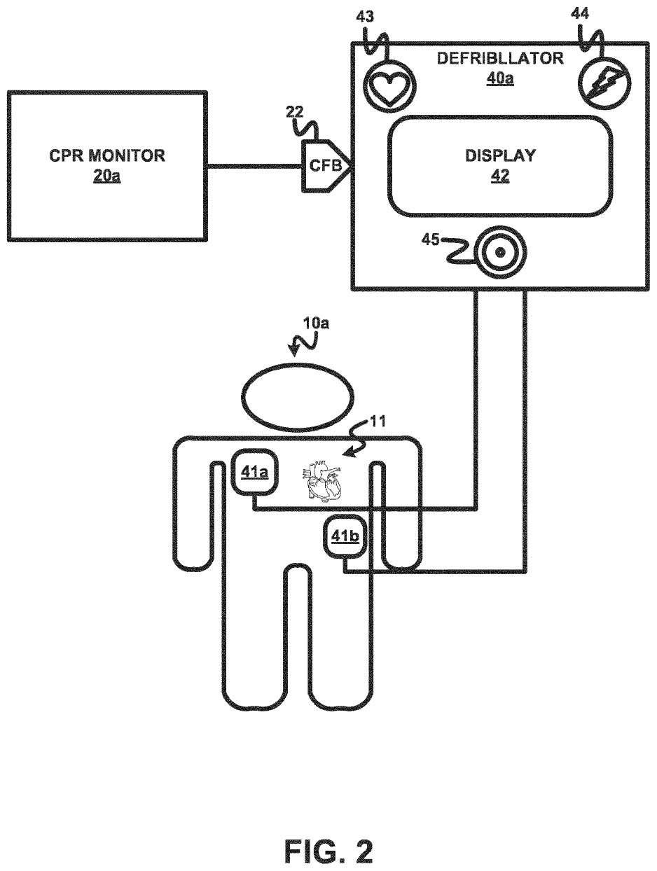

To facilitate an understanding of the present disclosure, the following description of FIG. 2 teaches an exemplary embodiment of a cardiac arrest treatment system in accordance with the present disclosure. From the description of FIG. 2, those having ordinary skill in the art of the present disclosure will appreciate how to apply the present disclosure to make and use additional embodiments of cardiac arrest treatment systems in accordance with the present disclosure.

Referring to FIG. 2, the exemplary cardiac arrest treatment system of the present disclosure employs a CPR monitor 20a and a defibrillator 40a.

In practice, CPR monitor 20a is any device, as known in the art of the present disclosure or hereinafter conceived, for analyzing a quality of CPR being administered by a responder (not shown) to a heart 11 of a patient 10a.

In a first exemplary embodiment, CPR monitor 20a is configured as a CPR coaching device in accordance with U.S. Pat. No. 8,532,765 B2 entitled “CPR Coaching Device with Reduced Sensitivity to Motion” to Ochs et al., the entirety of which is hereby incorporated by reference.

In a second exemplary embodiment, monitor 20a is a mechanical CPR device incorporating the CPR analyzing principles described in Ochs et al.

Still referring to FIG. 2, in practice, defibrillator 40a is any type of defibrillator, as known in the art of the present disclosure or hereinafter conceived, incorporating the inventive principles of the present disclosure for customizing a cardiac therapy treatment of heart 11 of patient 10a during CPR being administered by the responder to heart 11 of patient 10a.

In operation, defibrillator 40a inputs a CPR feedback 22 from CPR monitor 20a as a basis for an evaluation of a quality of the CPR being administered by the responder to heart 11 of patient 10a.

In one exemplary embodiment, CPR feedback 22 is an indicator of whether the CPR quality is effective or ineffective.

In a second exemplary embodiment, CPR feedback 22 includes data quantifying various CPR parameters (e.g., depth and rate of compressions, chest release and recoil, and placement of a responder's hands on a patient's chest) whereby defibrillator 40a decides if the data is indicating the CPR quality as being effective or ineffective.

Further in operation, defibrillator 40a monitors an ECG waveform of heart 11 of patient 10a via electrode 41a and 41b applied to patient 10a as known in the art of the present disclosure, and analyzes the ECG waveform to detect any shockable rhythm of heart 11 of patient 10a as known in the art of the present disclosure.

If defibrillator 40a detects a shockable rhythm of heart 11 of patient 10a, then defibrillator 40a determines a vitality of the detected shockable rhythm of heart 11 of patient 10a. In one exemplary embodiment, defibrillator 40a executes an estimation of a probability (0-100%) of return-of-rhythm (ROR) after a delivery of a shock to heart 11 of patient 10a as known in the art of the present disclosure.

Thereafter, defibrillator 40a derives a treatment advisory from an analytical combination of the CPR quality and the shockable rhythm vitality to thereby instruct the responder for an optimal CPR/shock treatment of heart 11 of patient 10a.

In one exemplary embodiment, defibrillator 40a implements a decision matrix of the CPR quality as effective or ineffective and of the shockable rhythm vitality as probable ROR or improbable ROR.

For example, if the CPR quality is deemed effective, and a vitality score indicates an improbable ROR, then the treatment advisory will instruct the responder to continue the CPR on heart 11 of patient 10.

By further example, if the CPR quality is deemed ineffective, and a vitality score indicates an improbable ROR, then the treatment advisory will instruct the responder to modify the CPR on heart 11 of patient 10 in a manner to increase the effectiveness of the CPR.

Also by example, if the vitality score indicates a probable ROR, then the treatment advisory will instruct the responder to interrupt the CPR on heart 11 of patient 10a and immediately implement a shock protocol for heart 11 of patient 10a.

In practice, other combinations of CPR quality/shockable rhythm vitality and related information can be additional factor(s) in deriving the treatment advisory including, but not limited to, a length of time heart 11 of patient 10a is in ventricular fibrillation (VF). For example, if heart 10 of patient 11 has been in VF for at least three (3) minutes, then the treatment advisory may instruct the responder to interrupt the CPR on heart 11 of patient 10a and immediately implement a shock protocol for heart 11 of patient 10a.

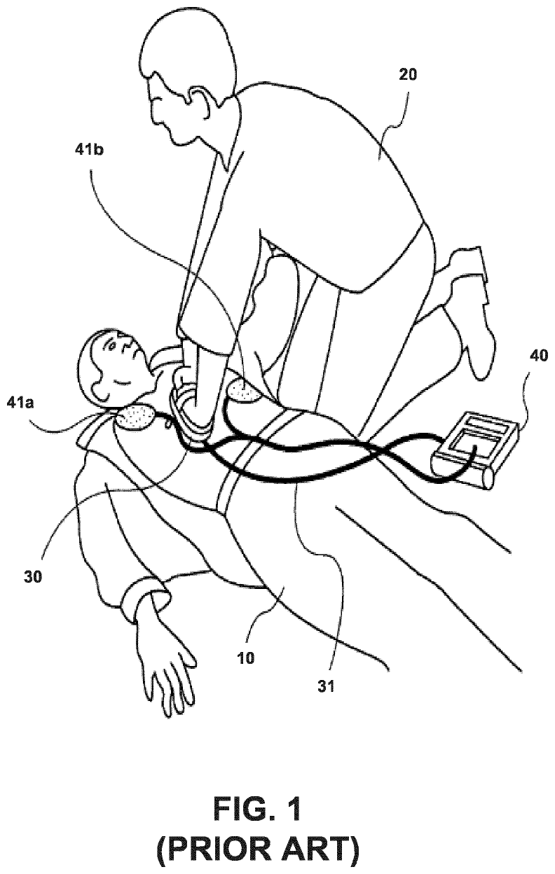

Still referring to FIG. 1, defibrillator 40a incorporates a display 42, a CPR advisory 43, a shock advisory 44, and a speaker 45 as means for communicating the treatment advisory to the responder.

For example, defibrillator 40a can communicate treatment advisory via display 42, CPR advisory 43 and/or speaker 45 to the responder to thereby instruct the responder to a continue, modify or interrupt the CPR being administered by the responder to the heart of the patient.

By further example, defibrillator 40a can communicate treatment advisory via display 42, shock advisory 44 and/or speaker 45 to the responder to thereby instruct the responder to execute shock protocol.

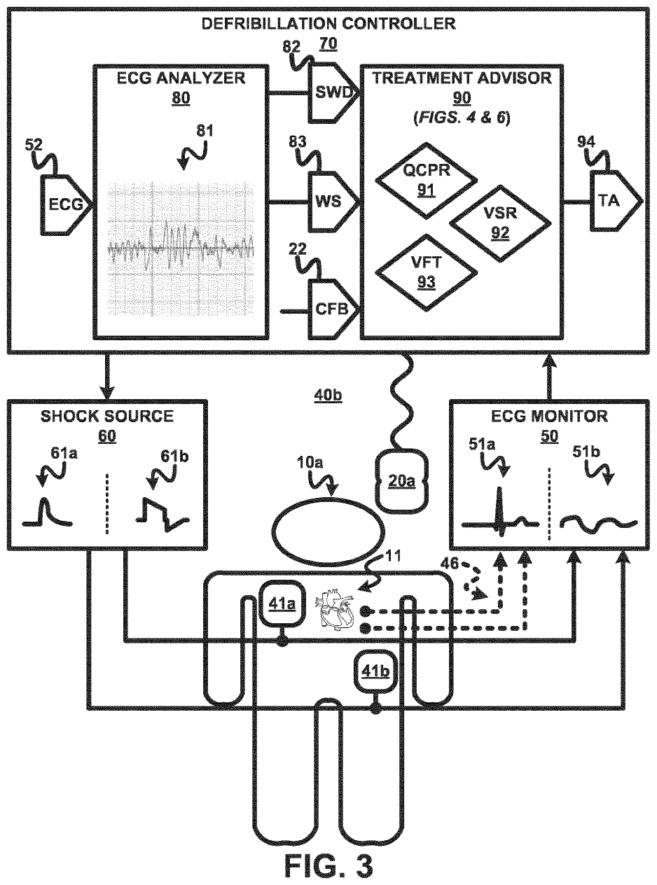

To further facilitate an understanding of the present disclosure, the following description of FIG. 3 teaches an exemplary embodiment of a defibrillator in accordance with the present disclosure. From the description of FIG. 3, those having ordinary skill in the art of the present disclosure will appreciate how to apply the present disclosure to make and use additional embodiments of defibrillator in accordance with the present disclosure.

Referring to FIG. 3, a defibrillator 40b of the present disclosure employs a pair of electrode pads/paddles 41a and 41b, optional ECG leads 46, an ECG monitor 50 (internal or external), a shock source 60, a defibrillation controller 70. Also shown is a CPR coaching device 20a communicatively coupled to defibrillation controller 70.

Electrode pads/paddles 41a and 41b are structurally configured as known in the art of the present disclosure to be conductively applied to a patient 10a in an anterior-apex arrangement as shown in FIG. 1 or alternatively in an anterior-posterior arrangement (not shown). Electrode pads/paddles 41a and 41b conduct a defibrillation shock from shock source 60 to heart 11 of patient 10a as controlled by defibrillation controller 70 as known in the art of the present disclosure, and conduct electrical activity of heart 11 of patient 10a to ECG monitor 50 as known in the art of the present disclosure. Alternatively or concurrently, ECG leads 46 as known in the art of the present disclosure may be connected to patient 10a to conduct the electrical activity of heart 11 of patient 10a to ECG monitor 50.

ECG monitor 50 is structurally configured as known in the art to generate an ECG waveform of heart 11 of patient 10a as an indication patient 10a is experiencing an organized heartbeat condition or an unorganized heartbeat condition. An example of ECG waveform indicating an organized heartbeat condition is an ECG waveform 51a as shown in FIG. 3 that is representative of an organized contraction of the ventricles of heart 11 being capable of pumping blood. An example of ECG waveform indicating patient 10a is experiencing an unorganized heartbeat condition is a random ECG waveform 51b as shown in FIG. 3 having zero (0) discernible waves representative of no organized heartbeat activity of heart 11 of patient 10a.

In one exemplary embodiment, ECG monitor 50 employs a digital signal processor (not shown) for streaming ECG waveform data 52 to defibrillation controller 70.

Shock source 60 is structurally configured as known in the art of the present disclosure to store electric energy for delivery of a defibrillation shock via electrode pads/paddles 41a and 41b to heart 11 of patient 10a as controlled by defibrillation controller 70. In practice, the defibrillation shock may have any waveform as known in the art of the present disclosure.

Examples of such waveforms include, but are not limited to, a monophasic sinusoidal waveform (positive sine wave) 61a and a biphasic truncated waveform 61b as shown in FIG. 3.

In one exemplary embodiment, shock source 60 employs a high voltage capacitor bank (not shown) for storing a high voltage via a high voltage charger and a power supply upon a pressing of a charge button. Shock source 60 further employs a switching/isolation circuit (not shown) for selectively applying a specific waveform of an electric energy charge from the high voltage capacitor bank to electrode pads/paddles 41a and 41b as controlled by defibrillation controller 70.

Defibrillation controller 60 incorporates an ECG analyzer 80, as known in the art of the present disclosure and hereinafter conceived, for analyzing and interpreting ECG waveform data 52 from ECG monitor 50.

Defibrillation controller 60 further incorporates a treatment advisor 90 to customize a cardiac therapy treatment of a patient during a cardiopulmonary resuscitation (CPR) being administered by a responder to a heart of the patient by deriving and communicating a treatment advisory from an evaluation of a quality of the CPR by the responder (e.g., in terms of depth and rate of compressions, chest release and recoil, and placement of a responder's hands on a patient's chest) in combination with a determination of a vitality of a detected shockable rhythm of the heart of the patient (e.g., ventricular fibrillation (VF) or ventricular tachycardia (VT)).

In one exemplary embodiment, defibrillation controller 70 encompasses all structural configurations, as understood in the art of the present disclosure and hereinafter conceived, of a main circuit board or an integrated circuit for controlling an application of various inventive principles of the present disclosure as exemplary described in the present disclosure. The structural configuration of defibrillation controller 70 may include, but is not limited to, processor(s), computer-usable/computer readable storage medium(s), an operating system, application module(s), peripheral device controller(s), slot(s) and port(s).

Also in the exemplary embodiment,. ECG analyzer 80 and treatment advisor 90 broadly encompasses an application incorporated within or accessible by a defibrillation controller 70 consisting of an electronic circuit (e.g., electronic components and/or hardware) and/or an executable program (e.g., executable software stored on non-transitory computer readable medium(s) and/or firmware) for executing a specific application.

In operation, ECG analyzer 80 will communicate a shockable rhythm detection 82 to treatment advisor 90 upon detecting a shockable rhythm in the ECG waveform, such as, for example, a ventricular fibrillation 81 as shown in FIG. 3. ECG analyzer 80 will further communicate a segment 83 of the shockable rhythm in the ECG waveform.

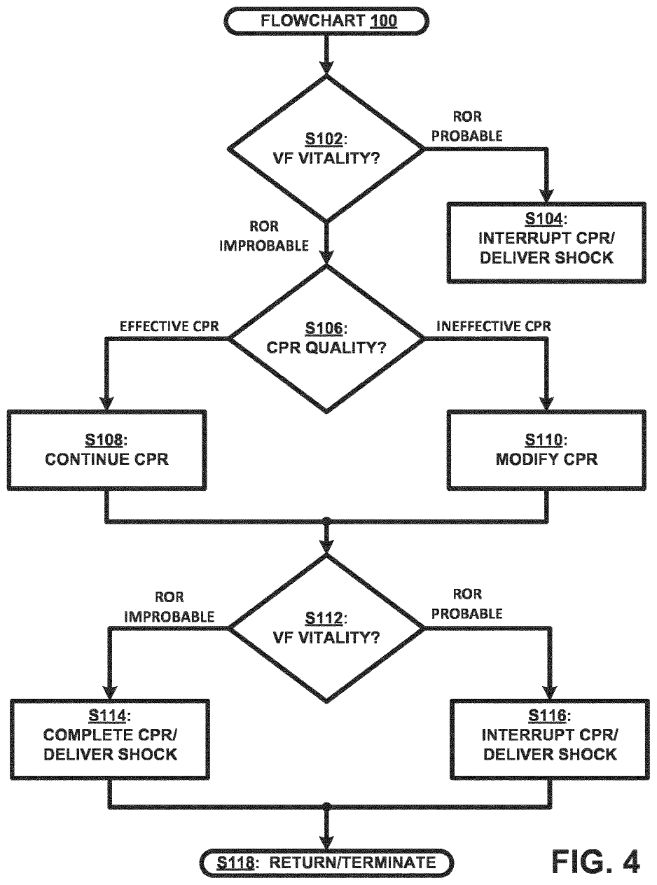

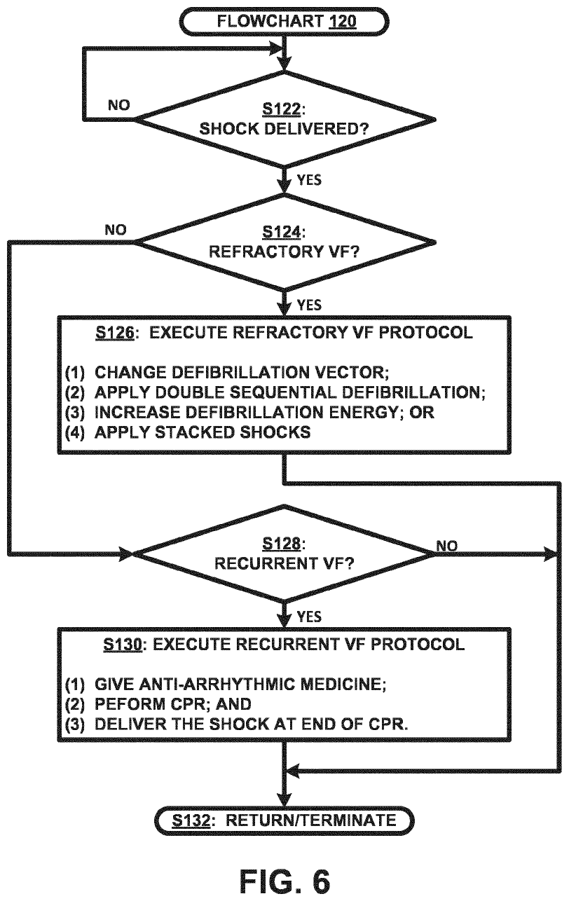

In response thereto, treatment advisor 90 will generate a vitality score for the shockable rhythm of the ECG waveform and will interpret CPR feedback 22 from CPR coaching device 20a to enable treatment advisor 90 to implement a flowchart 100 (FIG. 4) and a flowchart 120 (FIG. 6) representative of a cardiac arrest treatment method of the present disclosure for communicating treatment advisories 94 to users of defibrillator 70.

FIGS. 4 and 6 will be described herein in the context of a VF detection in the ECG waveform. Nonetheless, those having ordinary skill in the art of the present disclosure will appreciate how to apply the principles of FIGS. 4 and 6 to other shockable rhythms.

Referring to FIG. 4, a stage S102 of flowchart 100 encompasses determining if the vitality of ventricular fibrillation 81 indicates a probable return of rhythm (ROR) or an improbable ROR of heart 11 of patient 10a.

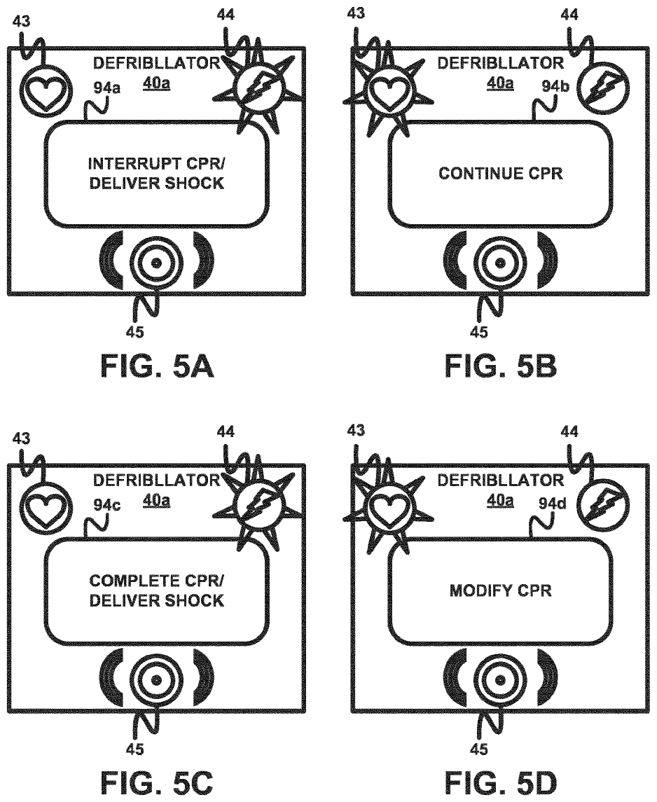

If treatment advisor 90 determines the vitality of ventricular fibrillation 81 indicates a probable return of rhythm (ROR) of heart 10 of patient 11a (e.g., the vitality score of ventricular fibrillation 81 is 75% or greater), then treatment advisor 90 proceeds to a stage S104 of flowchart 100 to derive an “Interrupt CPR/Deliver Shock” treatment advisory 94 a and to communicate treatment advisory 94a to the responder as exemplary shown in FIG. 5A.

Otherwise, if treatment advisor 90 determines the vitality of ventricular fibrillation 81 indicates an improbable return of rhythm (ROR) of heart 10 of patient 11a (e.g., the vitality score of ventricular fibrillation 81 is less than 75%), then treatment advisor 90 proceeds to a stage S106 of flowchart 100 to determine if the CPR quality indicates the CPR is effective or ineffective.

If treatment advisor 90 determines the CPR quality indicates the CPR is effective (e.g., depth and a rate of compression are within an acceptable range, chest release and recoil are within an acceptable range, and placement of the responder's hands on the chest of patient 10 is in an acceptable position), then treatment advisor 90 proceeds to a stage S108 of flowchart 100 to derive a “Continue CPR” treatment advisory 94b and to communicate treatment advisory 94b to the responder as exemplary shown in FIG. 5B.

Otherwise, if treatment advisor 90 determines the CPR quality indicates the CPR is ineffective (e.g., depth and a rate of compression are not within an acceptable range, chest release and recoil are nor within an acceptable range, and/or a placement of the responder's hands on the chest of patient 10 is not in an acceptable position), then treatment advisor 90 proceeds to a stage S110 of flowchart 100 to derive an “Modify CPR” treatment advisory 94c and to communicate treatment advisory 94c to the responder as exemplary shown in FIG. 5C. The “Modify CPR” treatment advisory 94c will include instructions on how to improve the effectiveness of the CPR quality.

Upon completion of stage S108 or stage S110, treatment advisor 90 proceeds to a stage S112 of flowchart 100 to determine if the vitality of ventricular fibrillation 81 is still indicating an improbable return of rhythm (ROR) or is now indicating a probable ROR of heart 11 of patient 10a.

If treatment advisor 90 determines the vitality of ventricular fibrillation 81 is still indicating an improbable return of rhythm (ROR) of heart 10 of patient 11a (e.g., the vitality score of ventricular fibrillation 81 is still less than 75%), then treatment advisor 90 proceeds to a stage S114 of flowchart 100 to derive “Complete CPR/Deliver Shock” treatment advisory 94d and to communicate treatment advisory 94d to the responder as exemplary shown in FIG. 5D.

Otherwise, if treatment advisor 90 determines the vitality of ventricular fibrillation 81 is now indicating a probable return of rhythm (ROR) of heart 10 of patient 11a (e.g., the vitality score of ventricular fibrillation 81 is now 75% or greater), then treatment advisor 90 proceeds to a stage S116 of flowchart 100 to derive “Interrupt CPR/Deliver Shock” treatment advisory 94a and to communicate treatment advisory 94a to the responder as exemplary shown in FIG. 5A.

In practice, related information can be additional factor(s) in deriving the treatment advisory including, but not limited to, a length of time heart 11 of patient 10a is in ventricular fibrillation (VF).

For example, stage S102 may encompass a determination that the vitality score must indicate a probable ROR for immediately preceding to stage S104 to communicate the “Interrupt CPU/Deliver Shock” treatment advisory.

By further example, stage S102 may encompass a detection of a low vitality score of VF 81 and the score cannot be improved even after high-quality CPR at stage S108, then the shock shall be delivered at stage S114 anyway.

Again by example, stage S102 may encompass a determination that the vitality score must indicate a probable ROR for a specified time period (e.g., three (3) minutes) before proceeding to stage S104 to communicate the “Interrupt CPU/Deliver Shock” treatment advisory.

By further example, stage S102 may encompass a detection of a non-shockable rhythm prior to a detection of the VF 81 and reduce the vitality score of VF 81 accordingly or immediately proceed to stage S106.

Flowchart 100 will terminate or return to stage S102 upon completion of stage S114 or stage S116.

FIG. 6 illustrates a flowchart 120 representative of an exemplary embodiment of a post-shock cardiac arrest treatment method of the present disclosure.

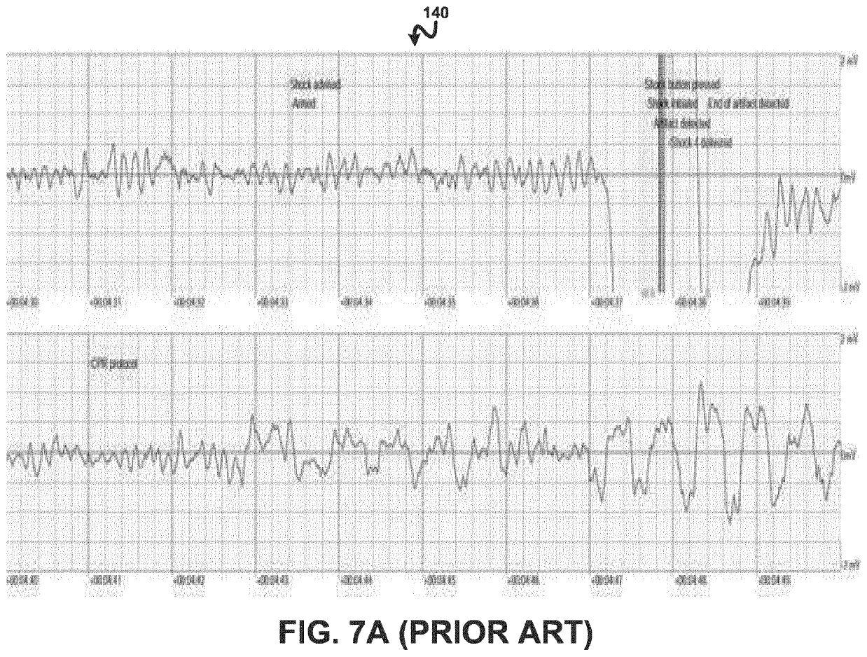

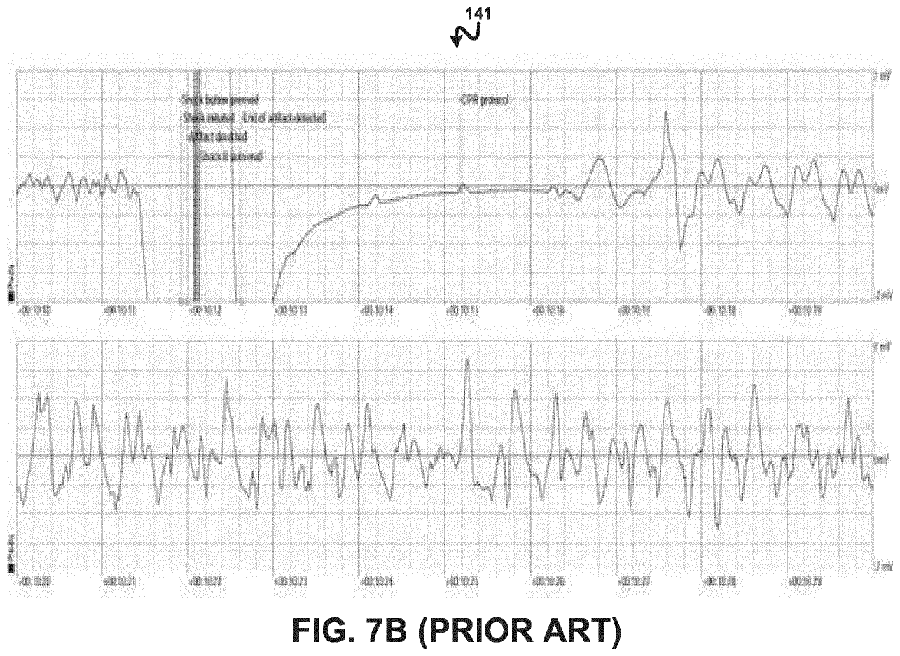

Referring to FIG. 6, upon a determination by treatment advisor 90 of a deliver shocked during a stage S122 of flowchart 120, a stage S124 of flowchart 120 encompasses treatment advisor 90 determining if a refractory VF is present in the ECG waveform. A refractory shock is an “unsuccessful shock” (i.e., VF continues post-shock). FIG. 7A illustrates an exemplary refractory VF 140 in an ECG waveform as known in the art of the present disclosure.

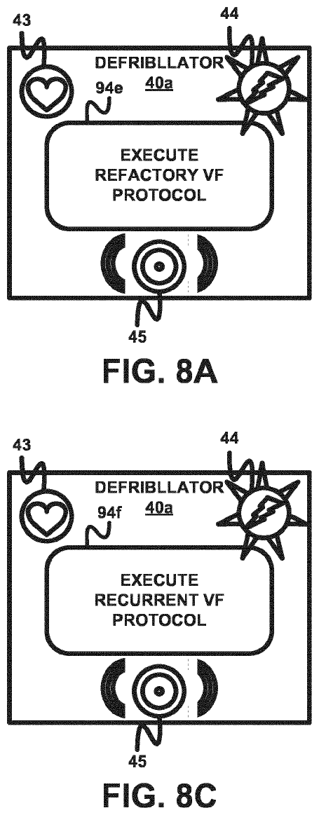

Still referring to FIG. 6, if treatment advisor 90 determines a refractory VF is present in the ECG waveform, then treatment advisor 90 proceeds to derive an “Execute Refractory VF Protocol” treatment advisory 94e and to communicate a treatment advisory 94e to the responder as exemplary shown in FIG. 8A. The treatment advisory 94e can include instructions to (1) change a defibrillation vector, (2) apply double sequential defibrillation, (3) increase defibrillation energy or (4) to apply stacked stocks.

Otherwise, if treatment advisor 90 determines a refractory VF is not present in the ECG waveform, then treatment advisor 90 proceeds to a stage S128 of flowchart 120 to determine if a recurrent VF is present in the ECG waveform. A recurrent shock is a “successful shock” that terminates the VF for a specified time period (e.g., 5 seconds), but heart 11 of patient 10a refibrillates. FIG. 7B illustrates an exemplary recurrent VF 141 in an ECG waveform as known in the art of the present disclosure.

If treatment advisor 90 determines a recurrent VF is present in the ECG waveform, then treatment advisor 90 proceeds to derive an “Execute Recurrent VF Protocol” treatment advisory 94f and to communicate treatment advisory 94f to the responder as exemplary shown in FIG. 8B. The treatment advisory 94f can include instructions to (1) give an anti-arrhythmic medicine to patient 10a, (2) perform CPR, and (3) deliver a shock at the end of the CPR.

Otherwise, if treatment advisor 90 determines a recurrent VF is not present in the ECG waveform, then treatment advisor 90 proceeds to a terminate flowchart 120 or return to stage S122.

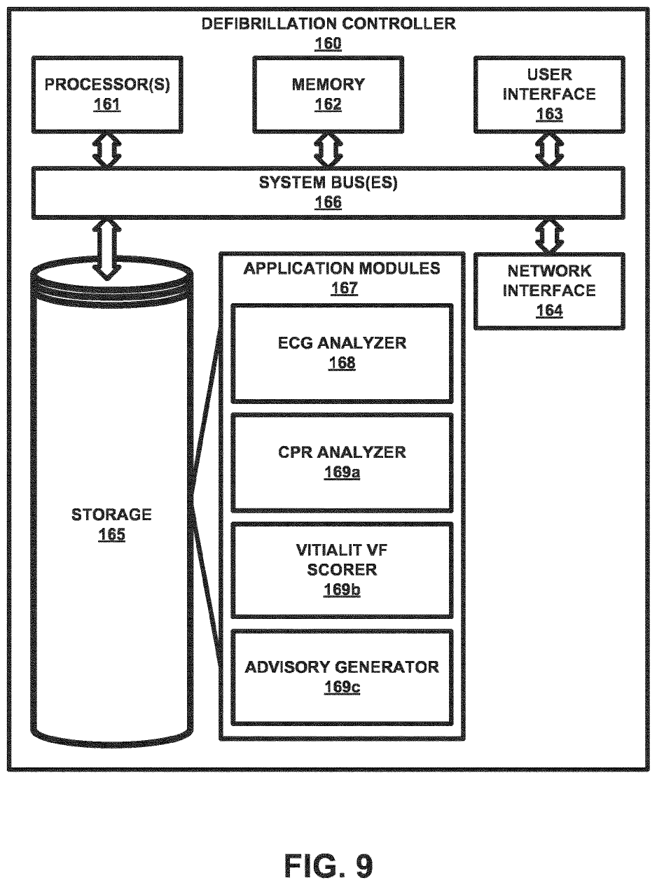

To facilitate a further understanding of the present disclosure, the following description of FIG. 9 teaches an exemplary embodiment of defibrillation controller in accordance with the present disclosure. From the description of FIG. 9, those having ordinary skill in the art of the present disclosure will appreciate how to apply the present disclosure to make and use additional embodiments of a defibrillation controller in accordance with the present disclosure.

Referring to FIG. 9, shown is an exemplary embodiment of defibrillation controller 160 that includes one or more processor(s) 161, memory 162, a user interface 163, a network interface 164, and a storage 165 interconnected via one or more system bus(es) 166.

Each processor 161 can be any hardware device, as known in the art of the present disclosure or hereinafter conceived, capable of executing instructions stored in memory 162 or storage or otherwise processing data. In a non-limiting example, the processor(s) 161 can include a microprocessor, field programmable gate array (FPGA), application-specific integrated circuit (ASIC), or other similar devices.

The memory 162 can include various memories, as known in the art of the present disclosure or hereinafter conceived, including, but not limited to, L1, L2, or L3 cache or system memory. In a non-limiting example, the memory 162 can include static random access memory (SRAM), dynamic RAM (DRAM), flash memory, read only memory (ROM), or other similar memory devices.

The user interface 163 can include one or more devices, as known in the art of the present disclosure or hereinafter conceived, for enabling communication with a user such as an administrator. In a non-limiting example, the user interface can include a command line interface or graphical user interface that can be presented to a remote terminal via the network interface 164.

The network interface 164 can include one or more devices, as known in the art of the present disclosure or hereinafter conceived, for enabling communication other components of a medical device. In a non-limiting example, the network interface 164 can include a network interface card (NIC) configured to communicate according to the Ethernet protocol.

Additionally, the network interface 164 may implement a TCP/IP stack for communication according to the TCP/IP protocols. Various alternative or additional hardware or configurations for the network interface 164 will be apparent.

The storage 165 can include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage 165 can store instructions for execution by the processor(s) 161 or data upon with the processor(s) 161 may operate. For example, the storage 165 may store a base operating system for controlling various basic operations of the hardware.

The storage 165 can also store an application modules in the form of executable software/firmware for implementing the various functions of the methods of FIGS. 4 and 6 as previously described in the present disclosure.

In one exemplary embodiment as shown, storage 165 stores application modules 167 including an ECG analyzer 168 for analyzing an ECG waveform to detect shockable rhythms as known in the art of the present disclosure, a CPR analyzer 169a for analyzing a quality of CPR being administered by a responder to a heart of a patient as known in the art of the present disclosure, and a vitality ventricular fibrillation scorer 169b for scoring a probability of a return of rhythm of a heart of a patient after a shock has been delivered to the heart of the patient as known in the art of the present disclosure.

Application modules 167 further includes an advisory generator 169c for deriving, in accordance with the present disclosure as previously described herein, a treatment advisory from the quality of CPR administered by a responder to a heart of a patient as analyzed by CPR analyzer 169a and from a probability score of a return of rhythm of a heart of a patient after a shock has been delivered to the heart of the patient as analyzed by vitality ventricular fibrillation scorer 169b.

From the description of FIGS. 1-9 herein, those having ordinary skill in the art will appreciate the numerous benefits of the present disclosure including, but not limited to, a customization of a cardiac arrest treatment of a patient facilitating optimal CPR administration and shock delivery to the patient.

The present disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

Further, as one having ordinary skill in the art shall appreciate in view of the teachings provided herein, features, elements, components, etc. disclosed and described in the present disclosure/specification and/or depicted in the appended Figures and/or recited in the Claims can be implemented in various combinations of hardware and software, and provide functions which may be combined in a single element or multiple elements. For example, the functions of the various features, elements, components, etc. shown/illustrated/depicted in the Figures and/or recited in the Claims can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and exemplary embodiments of the present disclosure, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar functionality, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of the present disclosure, which embodiments are intended to be illustrative and not limiting, it is noted that modifications and variations can be made by persons having ordinary skill in the art in view of the teachings provided herein, including the appended Figures and claims. It is therefore to be understood that changes can be made in/to the preferred and exemplary embodiments of the present disclosure which are within the scope of the present disclosure and exemplary embodiments disclosed, described and taught herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A defibrillator for customizing a cardiac therapy treatment of a patient during a cardiopulmonary resuscitation (CPR) being administered by a responder to a heart of the patient, the defibrillator comprising:
an ECG monitor configured to monitor an ECG waveform of the heart of the patient during the CPR being administered by the responder to the heart of the patient; and
a defibrillation controller,
wherein the defibrillation controller is configured to, when the ECG waveform as monitored by the ECG monitor indicates a shockable rhythm of the heart of the patient during the CPR being administered by the responder to the heart of the patient, derive a treatment advisory from at least a quality of the CPR being administered by the responder to the heart of the patient and a vitality of the shockable rhythm of the heart; and
wherein the defibrillation controller is further configured to customize the cardiac therapy treatment to the patient by communicating the treatment advisory to the responder.

2. The defibrillator of claim 1, wherein the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient.

3. The defibrillator of claim 2,
wherein, subsequent to the treatment advisory instructing the responder to interrupt the CPR being administered by the responder to the heart of the patient and to shock the patient, the treatment advisory instructs the responder to execute a refractory ventricular fibrillation protocol in response to the ECG waveform as monitored by the ECG monitor indicating a refractory ventricular fibrillation of the heart of the patient subsequent to the shock of the heart of the patient; and
wherein the refractory ventricular fibrillation protocol includes at least one additional shock to the heart of the patient.

4. The defibrillator of claim 2,
wherein, subsequent to the treatment advisory instructing the responder to interrupt the CPR being administered by the responder to the heart of the patient and to shock the patient, the treatment advisory instructs the responder to execute a recurrent ventricular fibrillation protocol in response to the ECG waveform as monitored by the ECG monitor indicating a recurrent ventricular fibrillation of the heart of the patient subsequent to the shock of the heart of the patient; and
wherein the recurrent ventricular fibrillation protocol includes a continuation or a modification of the CPR being administered by the responder to the heart of the patient followed by an additional shock to the heart of the patient.

5. The defibrillator of claim 1, wherein the treatment advisory instructs the responder to continue the CPR being administered by the responder to the heart of the patient in response to the vitality of the shockable rhythm of the heart of the patient indicating an improbable return-of-rhythm of the heart of the patient subsequent to a shock of the heart of the patient and in further response to the quality of the CPR being administered by the responder to the heart of the patient indicating an effective CPR being administered by a responder to the heart of the patient.

6. The defibrillator of claim 3, wherein, subsequent to the treatment advisory instructing the responder to continue the CPR being administered by the responder to the heart of the patient, the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient.

7. The defibrillator of claim 3, wherein, subsequent to the treatment advisory instructing the responder to continue the CPR being administered by the responder to the heart of the patient, the treatment advisory instructs the responder to complete the CPR being administered by the responder to the heart of the patient and to shock the patient in response to the vitality of the shockable rhythm of the heart of the patient indicating an improbable return-of-rhythm of the heart of the patient subsequent to the shock of the heart of the patient.

8. The defibrillator of claim 1, wherein the treatment advisory instructs the responder to modify the CPR being administered by the responder to the heart of the patient in response to the vitality of the shockable rhythm of the heart of the patient indicating an improbable return-of-rhythm of the heart of the patient subsequent to a shock of the heart of the patient and in further response to the quality of the CPR being administered by the responder to the heart of the patient indicating an ineffective CPR being administered by a responder to the heart of the patient.

9. The defibrillator of claim 6, wherein, subsequent to the treatment advisory instructing the responder to modify the CPR being administered by the responder to the heart of the patient, the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient.

10. The defibrillator of claim 6, wherein, subsequent to the treatment advisory instructing the responder to modify the CPR being administered by the responder to the heart of the patient, the treatment advisory instructs the responder to complete the CPR being administered by the responder to the heart of the patient and to shock the patient in response to the vitality of the shockable rhythm of the heart of the patient indicating an improbable return-of-rhythm of the heart of the patient subsequent to the shock of the heart of the patient.

11. A defibrillation controller, comprising:
a non-transitory machine-readable storage medium encoded with instructions for execution by at least one processor to adapt a cardiac therapy treatment of a patient during a cardiopulmonary resuscitation (CPR) being administered by a responder to a heart of the patient, the non-transitory machine-readable storage medium including the instructions to:
when an ECG waveform indicates a shockable rhythm of the heart of the patient during the CPR being administered by the responder to the heart of the patient, derive a treatment advisory from at least a quality of the CPR being administered by the responder to the heart of the patient and a vitality of the shockable rhythm of the heart of the patient; and
customize the cardiac therapy treatment to the patient by communicating the treatment advisory to the responder.

12. The defibrillation controller of claim 11, wherein the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient.

13. The defibrillation controller of claim 12,
wherein, subsequent to the treatment advisory instructing the responder to interrupt the CPR being administered by the responder to the heart of the patient and to shock the patient, the treatment advisory instructs the responder to execute a refractory ventricular fibrillation protocol in response to the ECG waveform as monitored by the ECG monitor (50) indicating a refractory ventricular fibrillation of the heart of the patient subsequent to the shock of the heart of the patient;
wherein the refractory ventricular fibrillation protocol includes at least one additional shock to the heart of the patient.
wherein, subsequent to the treatment advisory instructing the responder to interrupt the CPR being administered by the responder to the heart of the patient and to shock the patient, the treatment advisory instructs the responder to execute a recurrent ventricular fibrillation protocol in response to the ECG waveform as monitored by the ECG monitor indicating a recurrent ventricular fibrillation of the heart of the patient subsequent to the shock of the heart of the patient; and
wherein the recurrent ventricular fibrillation protocol includes a continuation or a modification of the CPR being administered by the responder to the heart of the patient followed by an additional shock to the heart of the patient.

14. The defibrillation controller of claim 11,
wherein the treatment advisory instructs the responder to continue the CPR being administered by the responder to the heart of the patient in response to the vitality of the shockable rhythm of the heart of the patient indicating an improbable return-of-rhythm of the heart of the patient subsequent to a shock of the heart of the patient and in further response to the quality of the CPR being administered by the responder to the heart of the patient indicating an effective CPR being administered by a responder to the heart of the patient;
wherein, subsequent to the treatment advisory instructing the responder to continue the CPR being administered by the responder to the heart of the patient, the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient; and
wherein, subsequent to the treatment advisory instructing the responder to continue the CPR being administered by the responder to the heart of the patient, the treatment advisory instructs the responder to complete the CPR being administered by the responder to the heart of the patient and to shock the patient in response to the vitality of the shockable rhythm of the heart of the patient indicating an improbable return-of-rhythm of the heart of the patient subsequent to the shock of the heart of the patient.

15. The defibrillation controller of claim 11,
wherein the treatment advisory instructs the responder to modify the CPR being administered by the responder to the heart of the patient in response to the vitality of the shockable rhythm of the heart of the patient indicating an improbable return-of-rhythm of the heart of the patient subsequent to a shock of the heart of the patient and in further response to the quality of the CPR being administered by the responder to the heart of the patient indicating an ineffective CPR being administered by a responder to the heart of the patient;
wherein, subsequent to the treatment advisory instructing the responder to modify the CPR being administered by the responder to the heart of the patient, the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient; and
wherein, subsequent to the treatment advisory instructing the responder to modify the CPR being administered by the responder to the heart of the patient, the treatment advisory instructs the responder to complete the CPR being administered by the responder to the heart of the patient and to shock the patient in response to the vitality of the shockable rhythm of the heart of the patient indicating an improbable return-of-rhythm of the heart of the patient subsequent to the shock of the heart of the patient.

16. A defibrillator method for customizing a cardiac therapy treatment of a patient during a cardiopulmonary resuscitation (CPR) being administered by a responder to a heart of the patient, the defibrillator comprising:
monitoring, by a defibrillator, a ECG waveform of the heart of the patient during the cardiopulmonary resuscitation being administered by the responder to the heart of the patient;
deriving, by the defibrillator when the ECG waveform as monitored by the defibrillator indicates a shockable rhythm of the heart of the patient during the CPR being administered by a responder to the heart of the patient, a treatment advisory from at least a quality of the CPR being administered by the responder to the heart of the patient and a vitality of the shockable rhythm of the heart of the patient; and
customizing the cardiac therapy treatment to the patient by the defibrillator (40a) communicating the treatment advisory to the responder.

17. The defibrillation method of claim 16, wherein the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient.

18. The defibrillation method of claim 17,
wherein, subsequent to the treatment advisory instructing the responder to interrupt the CPR being administered by the responder to the heart of the patient and to shock the patient, the treatment advisory instructs the responder to execute a refractory ventricular fibrillation protocol in response to the ECG waveform as monitored by the defibrillator indicating a refractory ventricular fibrillation of the heart of the patient subsequent to the shock of the heart of the patient;
wherein the refractory ventricular fibrillation protocol includes at least one additional shock to the heart of the patient.
wherein, subsequent to the treatment advisory instructing the responder to interrupt the CPR being administered by the responder to the heart of the patient and to shock the patient, the treatment advisory instructs the responder to execute a recurrent ventricular fibrillation protocol in response to the ECG waveform as monitored by the defibrillator indicating a recurrent ventricular fibrillation of the heart of the patient subsequent to the shock of the heart of the patient; and
wherein the recurrent ventricular fibrillation protocol includes a continuation or a modification of the CPR being administered by the responder to the heart of the patient followed by an additional shock to the heart of the patient.

19. The defibrillation method () of claim 16,
wherein the treatment advisory instructs the responder to continue the CPR being administered by the responder to the heart of the patient in response to the vitality of the shockable rhythm of the heart of the patient indicating an improbable return-of-rhythm of the heart of the patient subsequent to a shock of the heart of the patient and in further response to the quality of the CPR being administered by the responder to the heart of the patient indicating an effective CPR being administered by a responder to the heart of the patient;
wherein, subsequent to the treatment advisory instructing the responder to continue the CPR being administered by the responder to the heart of the patient, the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient; and
wherein, subsequent to the treatment advisory instructing the responder to continue the CPR being administered by the responder to the heart of the patient, the treatment advisory instructs the responder to complete the CPR being administered by the responder to the heart of the patient and to shock the patient in response to the vitality of the shockable rhythm of the heart of the patient indicating an improbable return-of-rhythm of the heart of the patient subsequent to the shock of the heart of the patient.

20. The defibrillation method of claim 16,
wherein, subsequent to the treatment advisory instructing the responder to modify the CPR being administered by the responder to the heart of the patient, the treatment advisory instructs the responder to interrupt the CPR being administered by the responder to the heart of the patient and to shock the patient in response to the vitality of the shockable rhythm of the heart of the patient indicating a probable return-of-rhythm of the heart of the patient subsequent to the shock of the heart of the patient; and
wherein, subsequent to the treatment advisory instructing the responder to modify the CPR being administered by the responder to the heart of the patient, the treatment advisory instructs the responder to complete the CPR being administered by the responder to the heart of the patient and to shock the patient in response to the vitality of the shockable rhythm of the heart of the patient indicating an improbable return-of-rhythm of the heart of the patient subsequent to the shock of the heart of the patient.
